(19) United States
(12) Patent Application Publication
(10) Pub. No.: US 2006/0058741 A1
(43) Pub. Date: Mar. 16, 2006

(54) Infusion apparatus for infusion bags

(76) Inventor: George Gallagher, Flintshire (GB)

Correspondence Address: CHARLES N. QUINN;FOX ROTHSCHILD LLP, 2000 MARKET STREET, 10TH FLOOR, PHILADELPHIA, PA, 19103, US

(21) Appl. No.: 10/538,026

(22) PCT Filed: Dec. 9, 2003
(86) PCT No.: PCT/GB03/05348

§ 371 Date: Jun. 8, 2005

(30) Foreign Application Priority Data
Dec. 9, 2002 (GB) 0228649.0

(51) Int. Cl. A61M 5/00

(52) U.S. Cl. 604/257; 604/246

(57) ABSTRACT

A modified giving set cap (40) to enable medication to be inserted into an intravenous bag (2) through the cap itself via a needleless syringe (5). The cap has a main channel (50) for delivering fluid from the bag/container (2) to a drip chamber (14) and a subsidiary channel for the delivery of a different material to the bag/container, the inlet (48) for delivering fluid from the hag through the main channel to the chamber being spaced at a sufficient distance from an outlet (46) of the subsidiary channel to allow mixing of the material with the fluid prior to delivery to the chamber. The inlet (45) of the subsidiary channel is adapted to receive a needleless syringe.

The present invention relates to an improved infusion apparatus, in particular in relation to the giving set of an infusion apparatus.

Healthcare workers are at risk from needlestick injuries from the use of needles and sharps. Not only are such injuries painful but these exposures can lead to the contraction of serious infections, such as hepatitis B, hepatitis C and Human Immunodeficiency Virus. This has led to a movement towards the replacement of a large number of needle devices by safer needleless systems. The majority of medications that were previously administered to a patient via the needle of a syringe may now be administered using a needle-free device. A large number of such devices of varying design are currently on the market. Generally medication is loaded into the syringe directly, without the requirement for a needle, by providing a luer adapter on the container containing the medication. The container is opened by applying pressure to a spring provided within the luer using the syringe tip. As soon as the syringe is removed from the luer, the opening is biased shut. The medication may then be administered to a patient by means of, for example an injector attached to the syringe that has substantially smaller hole than a needle.

Intravenous drip bags, for example for administering saline solution to a patient, often require medication, such as antibiotics, to be added to the bag. Currently, this is achieved by inserting the needle of a syringe through a self-sealable rubber bung provided at the base of the bag. Accordingly, it is still possible for healthcare staff to suffer needlestick injuries as a result of having to introduce fluid medicaments into an IV bag using a needle and syringe. This is clearly undesirable.

It is an object of the present invention to provide a novel giving set for an infusion apparatus that aims to overcome the abovementioned drawbacks.

Accordingly, a first aspect of the present invention provides a giving set cap for providing fluid communication between two vessels, the cap comprising a delivery tube having a main channel for delivering fluid from a first vessel to a second vessel and a subsidiary channel for the delivery of a different material to said first or second vessel characterised in that the cap includes one or both of the following features:
(a) an inlet for delivering fluid from said first or second vessel through the main channel, the inlet being spaced at a sufficient distance from an outlet of the subsidiary channel to allow mixing of the different material with said fluid prior to delivery to said other vessel; and
(b) an inlet of the subsidiary channel being adapted to receive a needleless syringe.

A second aspect of the present invention provides a giving set comprising a drip chamber attached to a line with a giving set cap as hereinbefore described.

A third aspect of the present invention provides an infusion apparatus comprising a IV bag or container, a giving set cap connecting said bag/container to a drip chamber having a line, characterised in that the giving set cap comprises a delivery tube having a main channel for delivering fluid from the bag/container to the chamber and a subsidiary channel for the delivery of a different material to said bag/container, the cap including one or both of the following features:
(a) an inlet for delivering fluid from the bag/container through the main channel to the chamber, the inlet being spaced at a sufficient distance from an outlet of the subsidiary channel to allow mixing of the different material with said fluid prior to delivery to the chamber; and
(b) an inlet of the subsidiary channel is adapted to receive a needleless syringe.

Preferably, the giving set cap further comprises connector means, the connector means allowing attachment of the cap to a giving set chamber. More preferably, the cap also comprises piercing means for attachment to an IV bag or container. Preferably, one end of the delivery tube is tapered to provide said piercing means. The main channel of the delivery tube preferably extends through said connector means.

It is preferable for the at least one inlet of the main channel to be provided in the part of the delivery tube that extends into the IV bag or container. Preferably, at least one outlet for the delivery tube is provided in the connector means. At least one inlet for the subsidiary channel is preferably provided in the connector means with at least one outlet being provided in the part of the delivery tube that extends into the IV bag or container, more preferably, being in or close to said piercing means.

Preferably, the giving set cap is provided with an inlet for delivering fluid from said first or second vessel through the main channel, the inlet being spaced at a sufficient distance from an outlet of the subsidiary channel to allow mixing of the different material with said fluid prior to delivery to said other vessel and is provided with a subsidiary channel that has an inlet that is adapted to receive a needleless syringe. In this manner, fluid can be inserted into the bag via the inlet of the subsidiary channel by means of a needleless syringe, enters the bag through an outlet of the subsidiary channel that is spaced at a sufficient distance from the inlet of the main channel to allow mixing of the different fluids prior to delivery to the chamber via the main channel.

Preferably, the outlet of the subsidiary channel is spaced at least 10 mm from the inlet of the main channel, more preferably still, the outlet is spaced at least 20 mm from the inlet of the main channel, especially at least 25 mm.

Preferably, the end of the piercing means is tapered but rounded. Preferably, the main channel is provided with at least two, preferably three inlets. Preferably, the subsidiary channel is provided with a single outlet.

The inlet of the subsidiary channel may also act as an air inlet to allow air to enter the bag via the giving cap. Preferably, the inlet is provided with a detachable cover. The inlet is preferably provided in one side of the connector means, preferably with the subsidiary channel extending up one side of the delivery tube.

In a preferred embodiment of the present invention, the inlet of the subsidiary channel is provided with an adapter having means for its temporary attachment to the inlet and having a passage therethough to act as an air vent, a cap being provided for closure of the air vent. In this manner, the adapter may be completely removed from the inlet to enable the delivery of material through the subsidiary channel or only the cap may be removed to allow the adapter to act as an air vent.

More preferably, the adapter has a male or female member for mating with a complimentary member on the inlet. More preferably, the adapter has an inner tube for inserting within the inlet and a threaded region for engagement with the outer sides of the inlet. Preferably, a membrane is provided across the passage of the adapter. More preferably, the cap is hingedly mounted with respect to the adapter. A vented Luer lock cap may be used as the adapter.

The connector means may be, for example, provided with internal threads for attachment to the drip chamber of a giving set. However, any other suitable means may be provided to allow attachment to the drip chamber via the connector means. Preferably, the connector means is cylindrical member having the main channel extending through the centre thereof.

For a better understanding of the present invention and to show more clearly how it may be carried into effect reference will now be made by way of example only to the accompanying drawings in which:

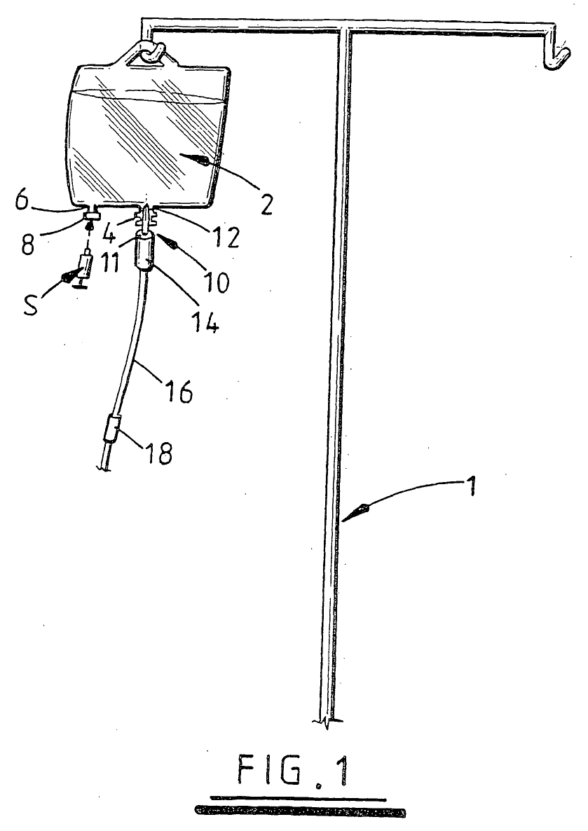

FIG. 1 is a schematic diagram of a conventional infusion apparatus;

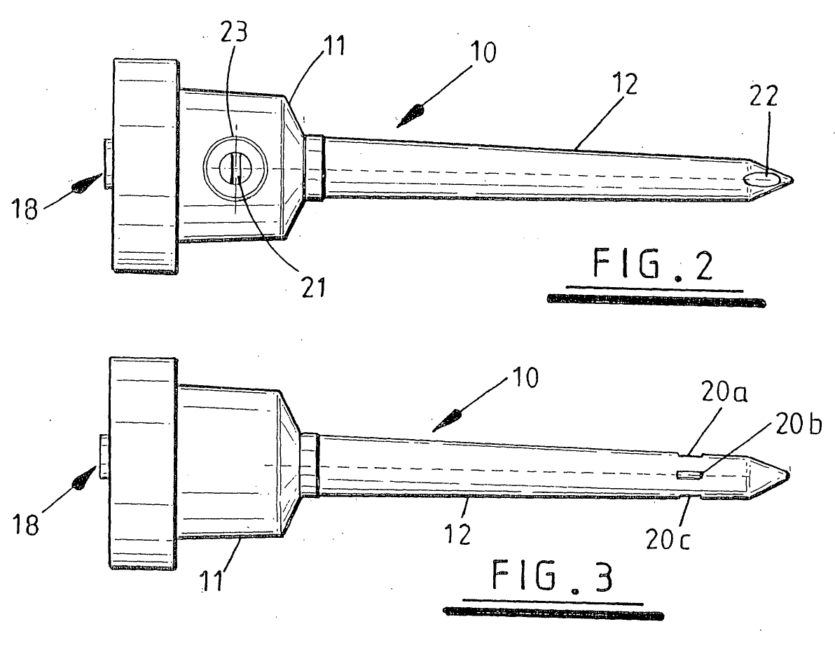

FIG. 2 is one side view of a conventional giving set cap used in the apparatus of FIG. 1;

FIG. 3 is the other side view of the giving set cap shown in FIG. 2;

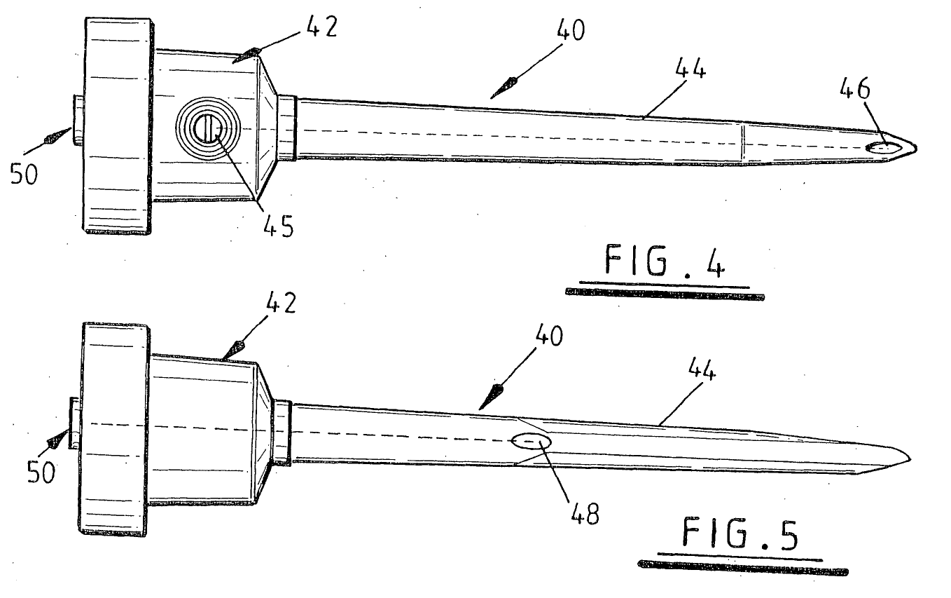

FIG. 4 is one side view of a giving set cap according to one embodiment of the present invention;

FIG. 5 is the other side view of the giving set cap shown in FIG. 4;

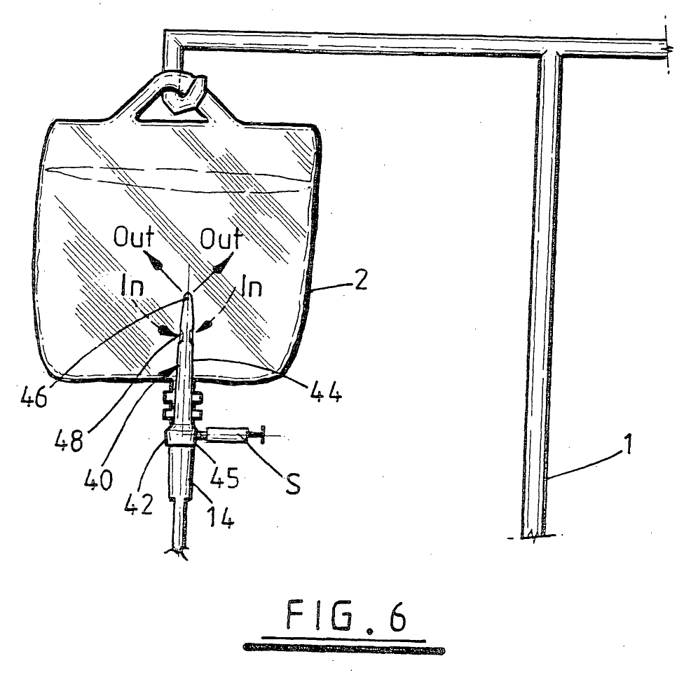

FIG. 6 is a schematic diagram of the giving set cap of FIGS. 4 and 5 attached to a conventional IV bag;

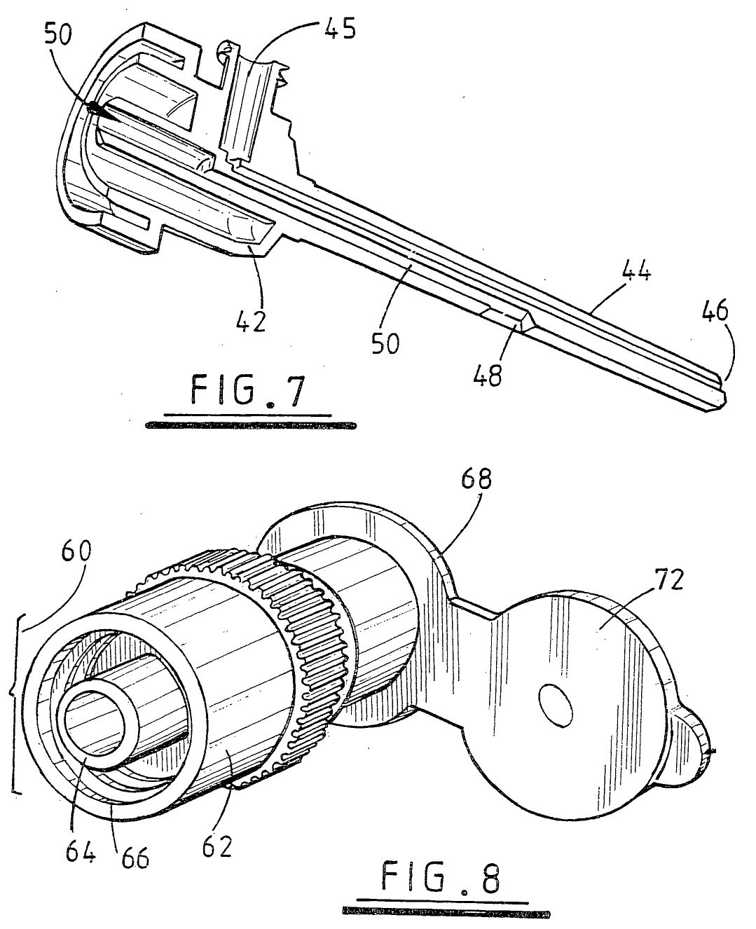

FIG. 7 is a perspective cross-sectional view of the giving set cap of FIGS. 4 and 5;

FIG. 8 is a perspective view of one type of adapter for attachment to a giving set cap according to the present invention;

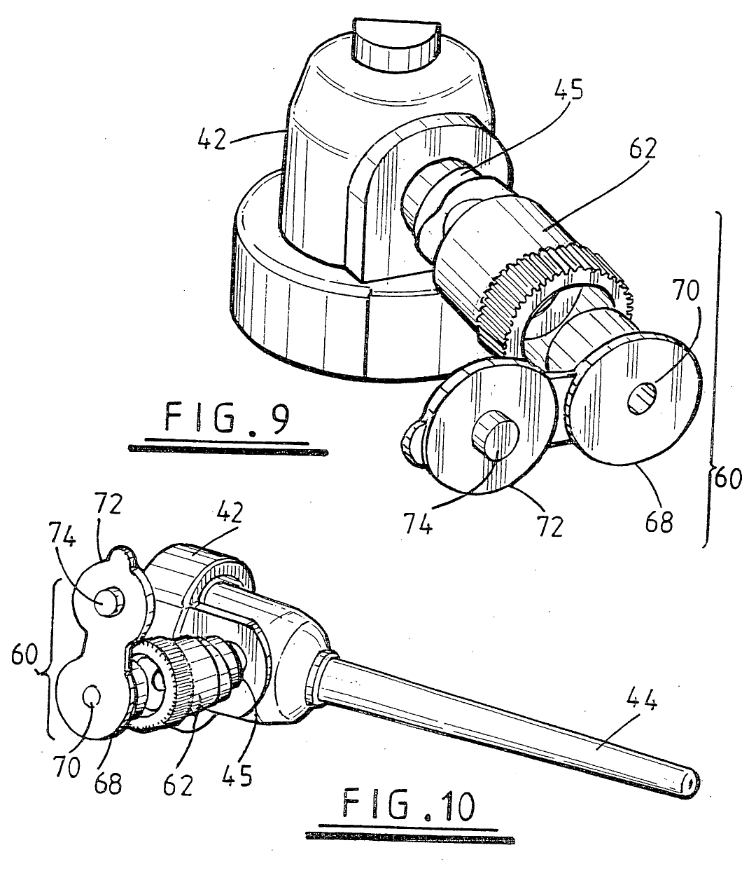

FIG. 9 is a perspective view illustrating the attachment of the adapter shown in FIG. 8 to a giving set cap; and

FIG. 10 is a perspective view of a giving set cap according to an embodiment of the present invention shown with an adapter.

FIGS. 1 to 3 of the accompanying drawings illustrate a conventional infusion apparatus, including the manner in which a solution of medication, such as an antibiotic, is introduced into an IV bag. The bag 2 is mounted on a stand 1 and the base of the bag is provided with a connector 4 and an outlet 6 sealed by means of a rubber bung 8. If a medicament is to be introduced into the bag, this is administered using a syringe with a needle S by inserting the needle through the rubber bung 8. The infusion line 16 is attached to the bag via a giving set cap 10. The giving set cap has a spike 12 that, in use, extends through the connector 4, and a luer 11 at the opposite end thereof that is secured to a drip chamber 14 that terminates in a line 16 that extends to a patient. A ball valve 18 enables the rate of flow of fluid through the line 16 to be adjusted. The giving set cap, drip chamber, line and valve makes up a giving set.

The spike 12 of the giving set cap 10 is provided with a central main channel 18 which terminates in three openings 20a, 20b, 20c on three sides of the spike towards the free end thereof, the opposite end of the channel extending past the end of the luer (see FIG. 3). Additionally, a second discrete channel 21 extends up one side of the spike to an opening 22 closer to the free end of the spike than the openings 20a, 20b, 20c (see FIG. 2). This channel extends from an air inlet 23 provided on one side of the luer of the cap that is normally provided with a cover (not shown). In this manner, fluid from the bag is able to flow through the openings 20a, 20b, 20c through the channel 18 and into the chamber to be administered to the patient. If the bag is of a rigid material, air is allowed to enter into the bag by removing the cover 25 from the cap to enable air to enter the inlet 23 flow up the channel 21 out of the opening 22 and into the bag, thereby preventing a vacuum forming above the fluid in the bag.

The present invention provides a modified giving set cap 40 to enable medication to be inserted into the bag through the cap itself via a needleless syringe, thereby removing the need for the separate outlet and the use of a needle. FIGS. 4, 5 and 7 of the accompanying drawings illustrate one embodiment of a giving set cap according to the present invention. The general construction of the cap is the same as hereinbefore described, i.e. having a luer end 42 for connection to a chamber and a spike 44 that may be inserted into the bag. However, the length of the spike of the cap is increased such that it is able to extend well into the body of the bag 2. The channel that extends from the air inlet 45 again extends to an opening 46 at or near to the free end of the spike but the openings 48 for the main channel 50 are distanced approximately 25 mm from the opening 46. Additionally, the inlet 45 is adapted to receive a needleless syringe and is provided with a cover (not shown). The head of the spike may be rounded so as to prevent puncturing of the bag. It is to be appreciated that the inlet may be adapted in any conventional manner depending upon the type of needleless syringe to be used with the giving set.

In this manner, a conventional needless syringe S may be used to introduce fluid into a bag 2 by engagement of the syringe with the adapted inlet, see FIG. 6. The fluid passes up and out of the upper outlet or opening 46, indicated by the unbroken arrows in FIG. 6. Fluid from the bag is still able to flow freely into the chamber 14 by means of the main channel 50 through openings 48, indicated by the broken arrows in FIG. 6. The distancing of the main channel from the side channel ensures that the medication is sufficiently diluted in the fluid contained in the bag before being delivered to a patient via the chamber and line. If a conventional giving set head was used, the medication introduced via the air inlet would pass straight out of the bag through the giving set in an undiluted state.

The present invention removes the requirement for a needle to be attached to a syringe to enable a fluid medicament to be introduced into an IV bag. This should assist in reducing the number of needlestick injuries suffered by medical staff and reduces the number of spent sharps that require disposal. Furthermore, it may also remove the need for the provision of additional site entry in the bag for receiving a needle. Mixing of the medicament with the fluid may also be enhanced since the medicament enters the bag at a higher level than the fluid exits the bag whereas with conventional sets the medicament enters the bag on the same level as the fluid exits the bag. The air inlet, though adapted to receive a needleless syringe, is still able to operate as valve to allow entry of air, when required, by removal of the cover.

FIG. 8 to 10 of the accompanying drawings illustrate a preferred giving set according to the present invention wherein the inlet 45 of the subsidiary channel is provided with an adapter 60. The adapter comprises a first part 62 for attachment to the inlet, having a central inner tube 64 for insertion within the inlet and a threaded region 66 for mating with the outer sides of the inlet. The second part of the adapter comprises a stopper 68 which is inserted within the first part, the stopper having a bore 70 through its centre with a membrane thereacross. The stopper is provided with a hingedly mounted cap 72 having a central knob 74 for insertion in the bore. In this manner, the adapter may be removed from the inlet to allow material to be into the subsidiary channel or only the cap may be removed to allow the adapter to act as an air vent.

1. A giving set cap for providing fluid communication between two vessels, the cap comprising a delivery tube having a main channel for delivering fluid from a first vessel to a second vessel and a subsidiary channel for the delivery of a different material to said first or second vessel, characterized in that the cap has an inlet for delivering fluid from said first or second vessel through the main channel, the inlet being spaced at a sufficient distance from an outlet of the subsidiary channel to allow mixing of the different material with said fluid prior to delivery to said other vessel and that an inlet of the subsidiary channel is adapted to receive a needleless syringe.

2. A giving set cap as claimed in claim 1, wherein the outlet of the subsidiary channel is spaced at least ten millimeters from the inlet of the main channel.

3. A giving set cap as claimed in claim 2 wherein the outlet of the subsidiary channel is spaced at least twenty millimeters from the inlet of the main channel.

4. (canceled)

5. A giving set cap as claimed in claim 1 further comprising connector means for allowing attachment of the cap to a giving set chamber.

6. A giving set cap as claimed in claim 1 further comprising piercing means for attachment of the cap to an intravenous bag or container.

7. A giving set cap as claimed in claim 6 wherein one end of the delivery tube is tapered to provide said piercing means.

8. (canceled)

9. A giving set cap as claimed in claim 5 wherein the main channel of the delivery tube extends through the connector means.

10. A giving set cap as claimed in claim 1 wherein the main channel is provided with at least two inlets.

11. A giving set cap as claimed in claim 10 wherein the main channel is provided with three inlets.

12. A giving set cap as claimed in claim 5 wherein at least one outlet for the delivery tube is provided in the connector means.

13. A giving set cap as claimed in claim 5 wherein at least one inlet 4 for the subsidiary channel is provided in the connector means.

14. A giving set cap as claimed in claim 1 wherein the inlet of the subsidiary channel is provided with a detachable cover.

15. A giving set cap as claimed in claim 1 wherein the inlet of the subsidiary channel is provided with an adapter having means for its temporary attachment to the inlet, a passage therethrough to act as an air vent and a detachable cap.

16. A giving set cap as claimed in claim 13 wherein the adapter has a male or female member for mating with a complementary member on the inlet.

17. A giving set cap as claimed in claim 14 wherein the adapter has an inner tube for inserting within the inlet and a threaded region for engagement with outer sides of the inlet.

18. A giving set cap as claimed in claim 15, wherein an air-permeable membrane is provided across the passage of the adapter.

19. A giving set cap as claimed in claim 15 wherein the cap is hingedly mounted with respect to the adapter.

20. A giving set cap as claimed in claim 13 wherein a vented Luer lock cap comprises the adapter.

21. A giving set cap as claimed in claim 5 wherein the inlet of the subsidiary channel is provided in one side of the connector means.

22. A giving set cap as claimed in claim 1 wherein the subsidiary channel extends up one side of the delivery tube.

23. A giving set cap as claimed in claim 5 wherein the connector means is provided with internal threads for attachment to a drip chamber of a giving set.

24. A giving set cap as claimed in claim 5 wherein the connector means comprises a cylindrical member having the main channel extending through the center thereof.

25. A giving set comprising a drip chamber attached to a line with a giving set cap as claimed in claim 5.

26. An infusion apparatus comprising an intravenous bag or container, a giving set cap connecting said bag/container to a drip chamber having a line characterised in that the giving set cap comprises a delivery tube having a main channel for delivery fluid from the bag/container to the chamber and a subsidiary channel for the delivery of a different material to said bag/container, the cap having a first inlet for delivery of fluid from the bag/container through the main channel to the chamber, and a second inlet for the subsidiary channel, the first inlet being spaced at a sufficient distance from an outlet of the subsidiary channel to allow mixing of the different material with said fluid prior to delivery to the chamber and the second inlet being adapted to receive a needleless syringe.

27. (canceled)

28. (canceled)

29. (canceled)

30. (canceled)

31. (canceled)

32. (canceled)

33. A giving set cap as claimed in claim 1 further comprising connector means for allowing attachment of the cap to a giving set chamber.
